(19) United States
(12) Patent Application Publication
(10) Pub. No.: US 2012/0123284 A1
(43) Pub. Date: May 17, 2012

(54) WIRELESS HEMODYNAMIC MONITORING SYSTEM INTEGRATED WITH IMPLANTABLE HEART VALVES

(76) Inventor: Arash Kheradvar, Irvine, CA (US)

(21) Appl. No.: 13/299,170

(22) Filed: Nov. 17, 2011

Related U.S. Application Data
(60) Provisional application No. 61/414,728, filed on Nov. 17, 2010.
(60) Provisional application No. 61/537,046, filed on Sep. 20, 2011.

(51) Int. Cl. A61B 5/02

(52) U.S. Cl. 600/509

(57) ABSTRACT

Described is a wireless hemodynamic monitoring system that is integrated with implantable cardiac devices. The system includes at least one sensory component that is adapted to measure one or more hemodynamic parameters inside a cardiac chamber of a subject. At least one transceiver is attached with the sensory component to transmit a signal containing data corresponding to the hemodynamic parameters and receive control signals from an external control device. An energy harvesting system is attached with the sensory component to measure pressures within the cardiac chamber and generate power for the monitoring system. The monitoring system can be attached with a heart valve or other cardiac device and implanted within a patient.

### PRIORITY CLAIM

This is a Non-Provisional Utility Patent Application of U.S. Provisional Application No. 61/414728, filed on Nov. 17, 2010, entitled, “Wireless Lab-on-a-Chip Apparatus for Implantable Cardiac Devices,” AND U.S. Provisional Application No. 61/537,046, filed on Sep. 20, 2011, entitled, “Wireless Lab on a Chip System Integrated with Implantable Heart Valves.”

### BACKGROUND OF THE INVENTION

(1) Field of Invention

The present invention relates to implantable cardiac devices and, more particularly, to a system assembly on a chip which can be implanted or integrated with an implantable cardiac device to monitor hemodynamic parameters.

(2) Description of Related Art

Heart failure affects nearly five million Americans. Roughly 550,000 people are diagnosed with heart failure each year, which is the leading cause of hospitalization in people older than the age of 65. Several techniques have been devised to diagnose heart related illnesses in an attempt to prevent heart failure. By way of example, there are a few modalities that are commonly used to measure hemodynamics, such as echocardiography (ultrasound) and cardiac catheterization.

Although ultrasound is widely-used, cheap, and non-invasive, it presents several drawbacks. In approximately 15% of the subjects tested with ultrasound, the results do not match the clinical observations. Further, use of ultrasound is limited in morbidly obese patients, it is operator-dependent, it does not provide continuous monitoring, and it is sensitive to acoustic shadow due to mechanical heart valves.

Alternatively, catheterization is the most accurate technique that provides a variety of measures, such as intracardiac pressure, systemic vascular resistance, blood O₂ content, and cardiac output. However, the catheterization procedure is invasive and relatively expensive. As was the case with the ultrasound procedure, catheterization does not provide any continuous monitoring. Importantly, catheterization is contraindicated in coagulopathy, hypertension, arrhythmia, fever, uncompensated heart failure, and mechanical heart valves.

Several recent innovations have been introduced in an attempt to overcome some of the issues presented by ultrasound and catheterization. A clinical study has shown that the EndoSure Wireless AAA Pressure Measurement System (i.e., implanted wireless monitoring device) has reduced hospitalization among heart failure patients by 39%. The EndoSure device is produced by CardioMEMS, Inc., located at 387 Technology Circle NW, Suite 500, Atlanta, Ga. 30313, U.S.A. The experimental implant is designed to measure pressure in a pulmonary artery, which is a leading indicator of how well a patient's heart failure is being managed with drugs.

As an alternative to the CardioMEMS device, a device called the HeartPod has a sensor that is implanted in the left atrial chamber of the heart. The HeartPod device is produced by Medtronic, Inc, located at 710 Medtronic Parkway, Minneapolis, Minn. 55432-5604, U.S.A. The HeartPod device measures the left atrial pressure, a highly consistent predictor of complications associated with chronic heart failure. The device also measures core temperature and the intracardiac electrogram, or EKG. The implanted device sends information to a hand-held unit that displays the measurements. More specifically, the HeartPod device includes a wire that goes into the heart, with a very small can which is used to extract the information. The wire and can are surgically implanted. Thereafter, a personal digital assistant (PDA) device is used to read fluid levels; telling the patient how much medicine to take. Through periodic monitoring using the HeartPod device, the dose of the patient's water pills can be adjusted to alleviate the congestion before the patient gets into trouble and ends up in the emergency department or hospital.

Medtronic also introduced the OptiVol Fluid Status Monitoring device. OptiVol monitoring provides clinicians an opportunity to assess the patient's fluid status via daily impedance measurements taken by an implantable cardiac resynchronization therapy defibrillator (CRT-D) or implantable cardioverter-defibrillator (ICD) device. Many times during the day, electrical impulses travel from the right ventricular lead to the implanted device can. OptiVol Fluid Status Monitoring uses this electrical impulse vector to measure impedance across the thoracic cavity. Trended daily impedance data has been shown to correlate well with pulmonary capillary wedge pressure (PCWP), pulmonary artery diastolic pressure (ePAD), and worsening heart failure.

While the devices listed above are suitable for their individual measuring parameters, they each require a specific procedure for implantation and, importantly, are limited by their measurement capabilities. Because Patients requiring valve repair or replacement are often presented initially with congestive heart failure and are at risk for future episodes of heart failure depending on their underlying function and volume status, it is desirable to provide such patients with an implantable wireless monitoring device to reduce the risk of future heart failure.

Thus, a continuing need exists for an implanted wireless monitoring device that that can be incorporated into an existing pre-approved heart-valve for monitoring blood flow rate, blood volume, and ventricular pressure, which can be used to enhance the clinical outcome of patients with aortic stenosis, leakage of the valves, coarctation of the aorta and pulmonary atresia.

### SUMMARY OF INVENTION

The present invention is a wireless monitoring system assembly on one or more chips which can be implanted or integrated with an implantable cardiac device to monitor hemodynamic parameters. The wireless monitoring system includes at least one sensory component adapted to measure one or more hemodynamic parameters inside a cardiac chamber of a subject; and at least one coordinator component that receives the data from the sensory component; and at least one transmitter communicatively attached with the coordinator component and adapted to transmit a signal containing data corresponding to the one or more hemodynamic parameters.

In one aspect, the at least one sensory component is integrated within an implantable cardiac device.

In yet another aspect, the sensory component is a microfluidic system that measures one or more hemodynamic parameter(s). The sensory component is a component selected from a group consisting of a magnetic probe, an ultrasonic probe, and a piezoelectric probe, and a flow measurement apparatus.

In another aspect, the implantable cardiac device is a device selected from a group consisting of a heart valve, an annuloplasty ring, and a mitral valve sewing ring.

Further, the transmitter is a transceiver that is adapted to both transmit a signal containing data corresponding to the one or more hemodynamic parameters and receive a control signal from an external control device.

In another aspect, an energy harvesting system is attached with the coordinator component.

Each of the sensory component, coordinator component, transceiver, and energy harvesting system are formed as micro-fabricated ring-form components and attached with one another in a stacked configuration. Alternatively, each of the sensory component, coordinator component, transceiver, and energy harvesting system can be formed as micro-fabricated components and attached with one another in a packed configuration.

In yet another aspect, at least one sensory component is adapted to measure a parameter selected from a group consisting of hydrostatic pressure of blood, blood gas partial pressures, blood velocity, blood viscosity, and cardiac chamber(s) volume. Additionally, the at least one sensory component configured to measure one or more hemodynamic parameters inside a cardiac chamber of a subject measures data intermittently or continuously, depending on the desired configuration.

In another aspect, a plurality of sensor/wireless communications devices are included for implantation near the surface of the patient. The sensor/wireless communications devices are operable for receiving a signal from the monitoring system and relaying the signal to other sensor/wireless communications devices or to an external monitor or network.

Additionally, the transmitter is configured to transmit data intermittently or continuously.

Finally, as can be appreciated by one in the art, the present invention also comprises a method for forming and using the invention described herein.

### BRIEF DESCRIPTION OF THE DRAWINGS

The objects, features and advantages of the present invention will be apparent from the following detailed descriptions of the various aspects of the invention in conjunction with reference to the following drawings, where:

FIG. 1 a cross-sectional view illustration of a heart with an artificial valve having a wireless hemodynamic monitoring system according to the present invention;

FIG. 2 is an illustration of an implantable device (artificial valve) having a wireless hemodynamic monitoring system according to the present invention to transmit and receive data to and from a receiver/transmitter disposed outside the body of a subject;

FIG. 3 is a schematic illustration the transmission of data from the system of the present invention for use and monitoring outside the body of the subject;

FIG. 4A is an illustration depicting the wireless hemodynamic monitoring system according to the present invention;

FIG. 4B is an illustration of a sensory component that is suitable for measuring blood flow and determining cardiac chamber volume;

FIG. 4C is an illustration depicting components of the energy harvesting system according to the present invention;

FIG. 5 is an illustration depicting components of the wireless hemodynamic monitoring system according to the present invention; and

FIG. 6 is an illustration depicting multi-hop communications according to the present invention.

### DETAILED DESCRIPTION

The present invention relates to implantable cardiac devices and, more particularly, to a system assembly on one or more chips which can be implanted or integrated with an implantable cardiac device to monitor hemodynamic parameters. The following description is presented to enable one of ordinary skill in the art to make and use the invention and to incorporate it in the context of particular applications. Various modifications, as well as a variety of uses in different applications will be readily apparent to those skilled in the art, and the general principles defined herein may be applied to a wide range of embodiments. Thus, the present invention is not intended to be limited to the embodiments presented, but is to be accorded the widest scope consistent with the principles and novel features disclosed herein.

In the following detailed description, numerous specific details are set forth in order to provide a more thorough understanding of the present invention.

However, it will be apparent to one skilled in the art that the present invention may be practiced without necessarily being limited to these specific details. In other instances, well-known structures and devices are shown in block diagram form, rather than in detail, in order to avoid obscuring the present invention.

The reader's attention is directed to all papers and documents which are filed concurrently with this specification and which are open to public inspection with this specification, and the contents of all such papers and documents are incorporated herein by reference. All the features disclosed in this specification, (including any accompanying claims, abstract, and drawings) may be replaced by alternative features serving the same, equivalent or similar purpose, unless expressly stated otherwise. Thus, unless expressly stated otherwise, each feature disclosed is only one example of a generic series of equivalent or similar features.

Furthermore, any element in a claim that does not explicitly state “means for” performing a specified function, or “step for” performing a specific function, is not to be interpreted as a “means” or “step” clause as specified in 35 U.S.C. Section 112, Paragraph 6. In particular, the use of “step of” or “act of” in the claims herein is not intended to invoke the provisions of 35 U.S.C. 112, Paragraph 6.

Please note, if used, the labels left, right, front, back, top, bottom, forward, reverse, clockwise and counter clockwise have been used for convenience purposes only and are not intended to imply any particular fixed direction. Instead, they are used to reflect relative locations and/or directions between various portions of an object.

Before describing the invention in detail, first an introduction provides the reader with a general understanding of the present invention. Thereafter, specific details of the present invention are provided.

(1) Introduction

Generally speaking, the present invention is related to implantable cardiac devices. While existing cardiac monitoring techniques and devices exist, they are either invasive, require expensive surveillance procedures, or do not provide continuous monitoring. Thus, the present invention is directed to an implanted wireless hemodynamic monitoring system that that can be incorporated into an existing, pre-approved heart-valve for continuously monitoring blood flow rate, blood volume, and ventricular pressure. Through such a device, healthcare costs will be considerably reduced by allowing physicians to monitor and titrate medical therapies based on hemodynamic data rather than just a physical exam. Obtaining such hemodynamic data generally requires noninvasive periodic studies such as echocardiograms or invasive right heart catheterization, whereas the present invention provides continuous hemodynamic data. Thus, the ability to more carefully monitor and fine-tune outpatient therapy will significantly reduce the healthcare cost of hospitalizations for congestive heart failure by preventing such occurrences.

(2) Specific Details

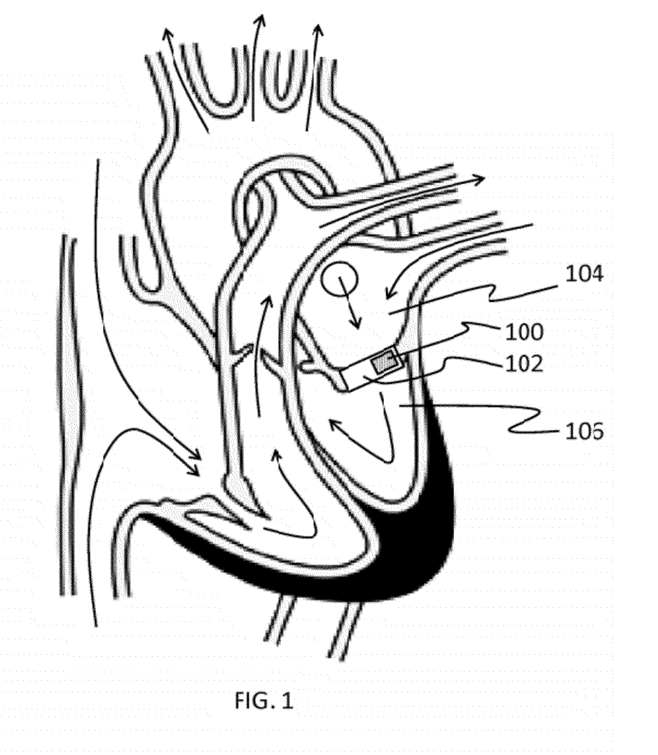

As shown in FIG. 1, the present invention describes a wireless hemodynamic monitoring system 100 that is implantable or integratable within or on an implantable cardiac device 102. Non-limiting examples of such an implantable cardiac device 102 include heart valves, annuloplasty rings, mitral valve sewing rings, or the like. In the example depicted in FIG. 1, the monitoring system 100 is attached with a heart valve (implantable cardiac device 102) that is positioned between the left atrium 104 and left ventricle 106. Other non-limiting examples of such heart valves include the Regent Valve, Hall Easyfit Carbon disc Valve, Bicarbon fitline, Carbomedics Top-hat Supraannular aortic valve.

The monitoring system 100 is capable of real-time sensing of hemodynamic parameters, non-limiting examples of which include hydrostatic pressure, blood oxygen/carbon dioxide partial pressures, blood velocity (i.e., blood flow rate), blood viscosity, blood biochemistry, etc., depending on the need of the patient. In one aspect, the monitoring system 100 can monitor the parameters related to functionality of the implanted cardiac device (such as a heart valve) that it is integrated with.

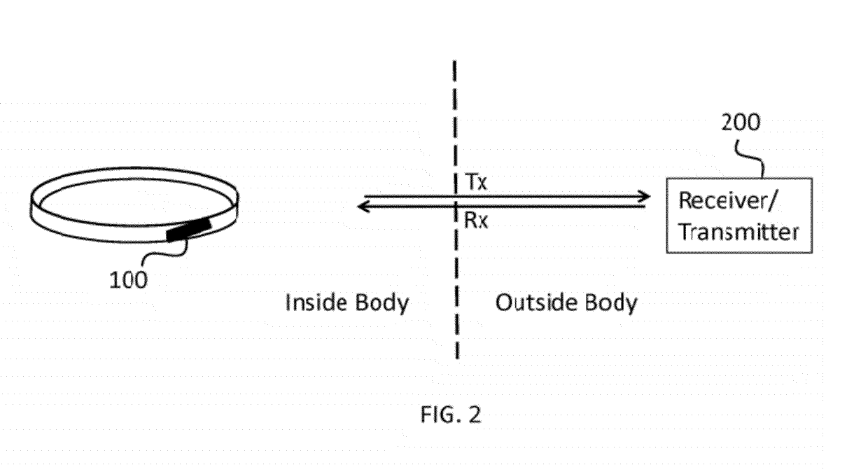

As shown in FIG. 2, the monitoring system 100 includes an integrated wireless transmitter that is operable for transmitting the measured data to a receiver 200 located outside the body.

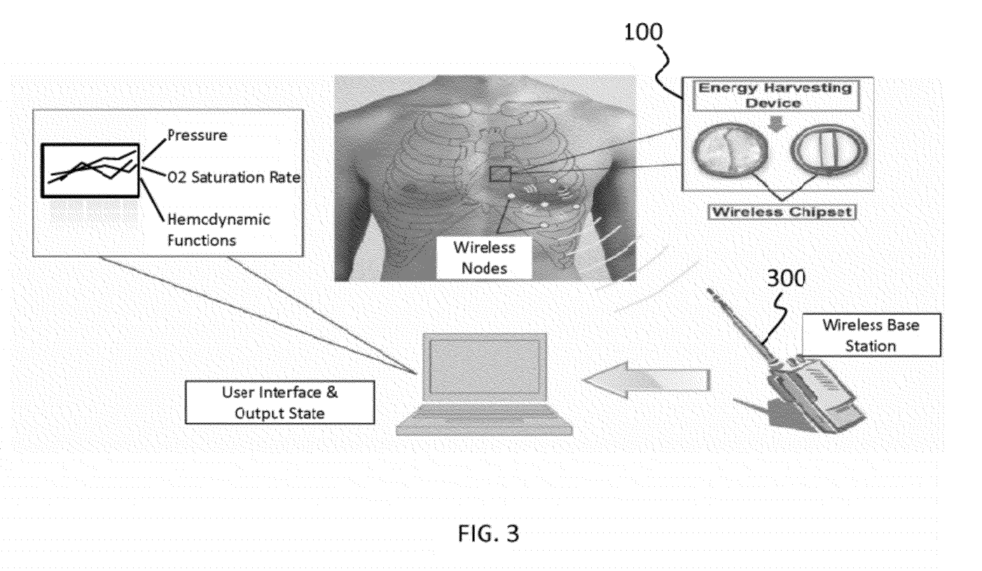

As shown in FIG. 3, the monitoring system 100 may incorporate a wireless transmitter or a separate transmitter may be associated with the device. For example, the transmission of the signals can be performed using a wireless sensor network (WSN) consisting of spatially distributed, autonomous, wireless communications devices (e.g., nodes) implanted within the body to transmit the data more effectively (as described in further detail below). Upon transmission of the hemodynamic data, a wireless base station 300 receives the data for further analysis by a user or clinician.

The monitoring system 100 can be internally powered through power sources such as but not limited to batteries (which may be re-chargeable through magnetic induction or the like) or the monitoring system 100 can utilize the body/blood thermal energy as a source of energy to drive the device or charge the device.

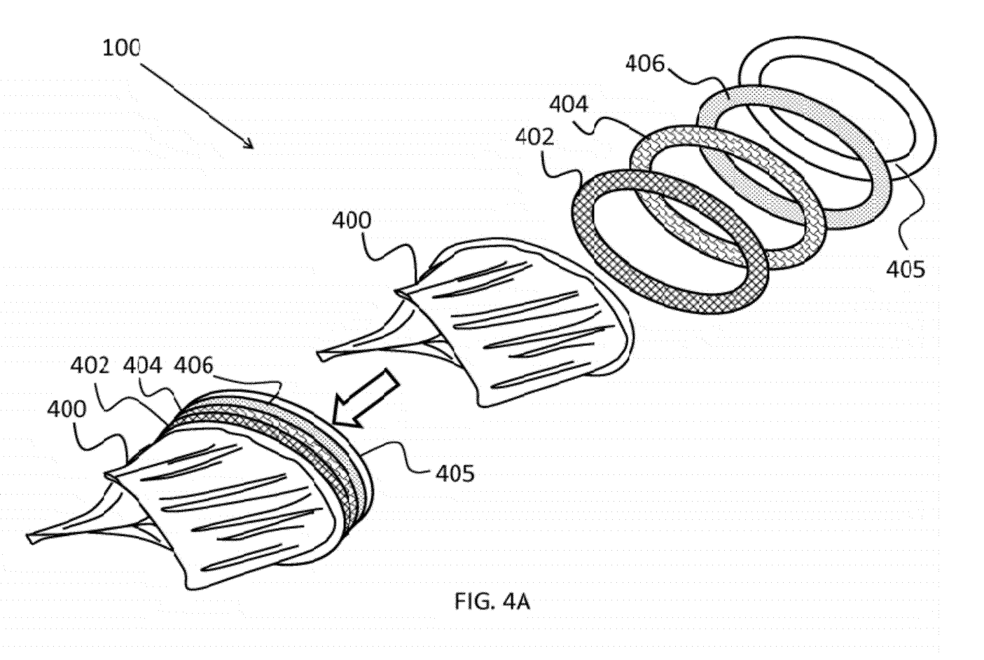

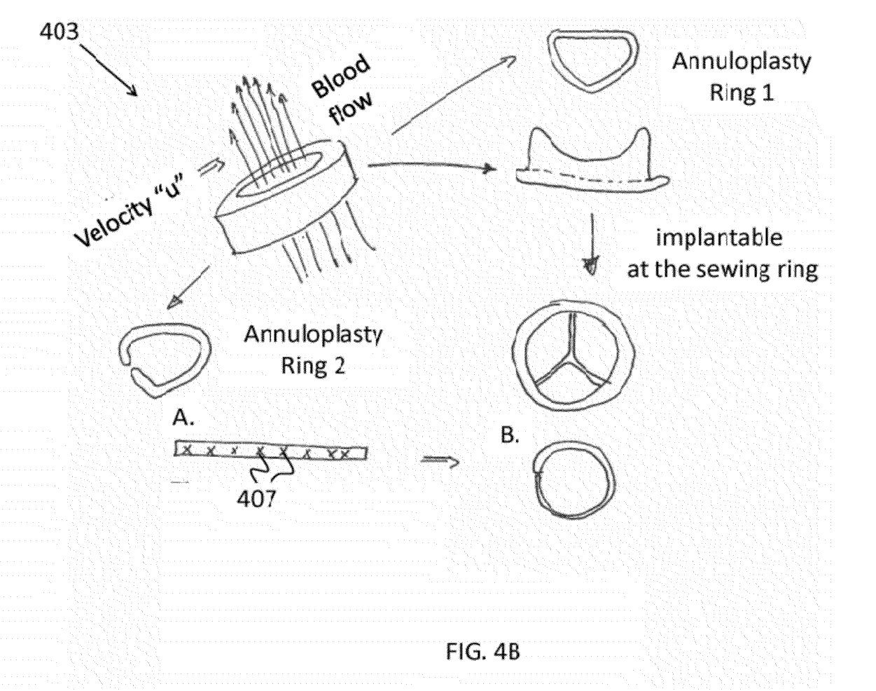

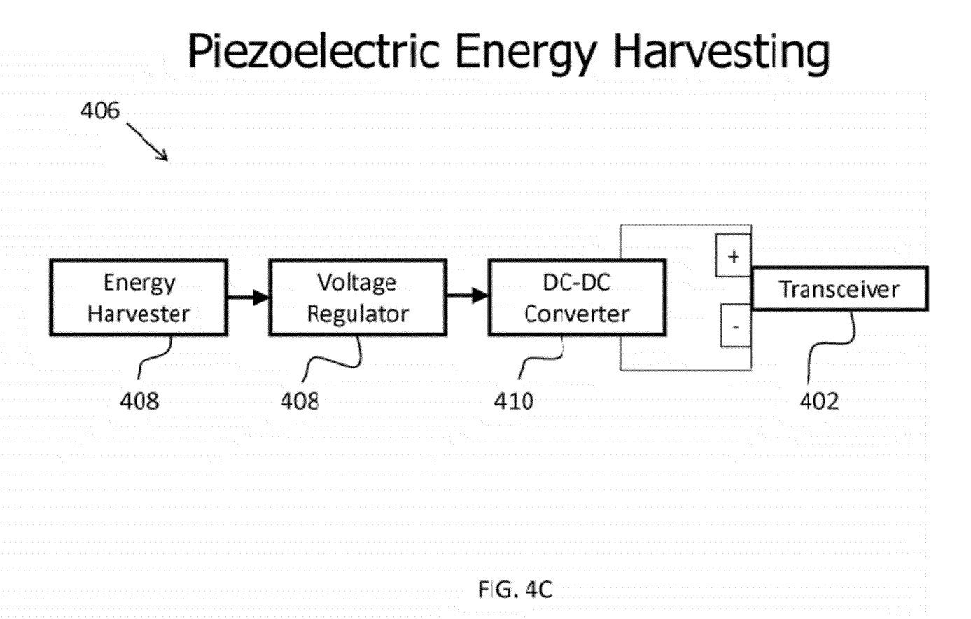

For further understanding, FIG. 4A illustrates a wireless hemodynamic monitoring system 100 according to the present invention. As shown, the system 100 includes a variety of lab-on-chip items that can be integrated with an implantable cardiac device (e.g., heart valve 400). For example, the system 100 includes a transceiver 402 (i.e., wireless circuit), a sensory component 404, a coordinator component 405, and an energy harvesting system 406, each of which can be formed as micro-fabricated ring-form components and attached with one another in a stacked configuration. Alternatively, each of the components can be formed in a packed configuration and formed into a single device.

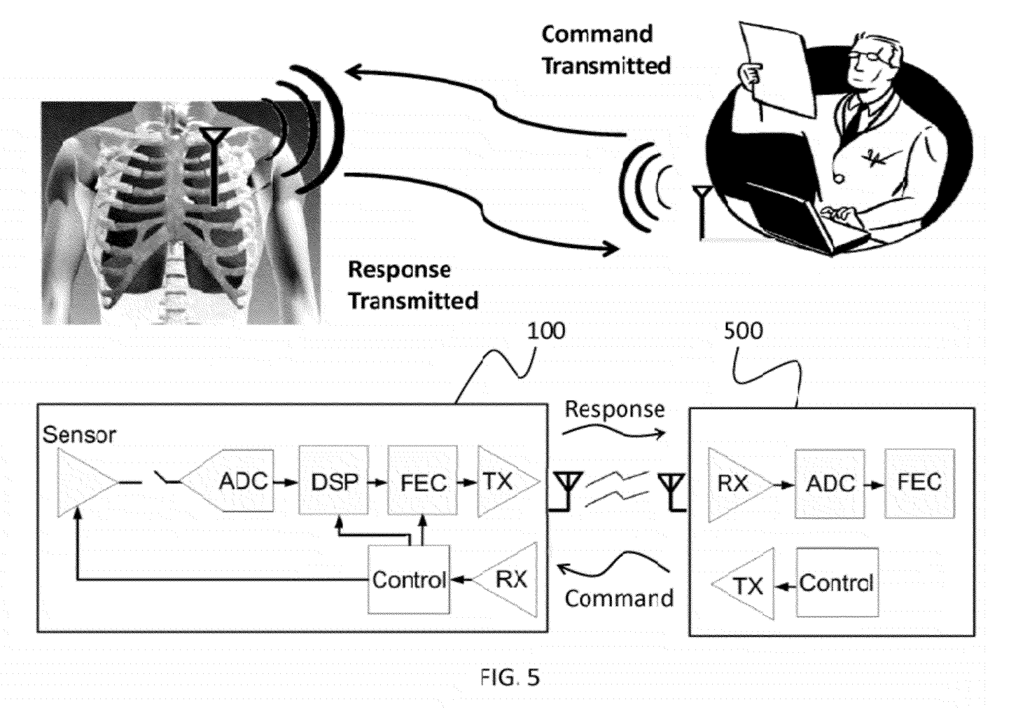

Each of the transceiver 402, sensory component 404 and energy harvesting system 406 can be incorporated into a single chip or multiple chips or any other suitable circuit or device. As a non-limiting example and as illustrated in FIG. 4A, the transceiver 402, sensory component 404 and energy harvesting system 406 are ring-shaped circuits/devices that are adapted to attach with the implantable cardiac device (e.g., heart valve 400). Further, to receive and coordinate the communications between the various components, a coordinator component 405 can be included. The coordinator component 405 is a circuit (e.g., integrated circuit) or chip that receives data from the sensory components 404 and energy harvesting/piezoelectric measurement system 406 and provides the data to the transceiver 402. Alternatively, any data received from the transceiver 402 is processed through the coordinator component 405, with commands distributed appropriately. Thus, the coordinator component 405 operates as a micro-processor. The coordinator component 405 can optionally include a digital signal processor, a control, and a forward error correction system (as illustrated in FIG. 5) to control and process the various signals. Further the coordinator component 405 can be formed as a separate circuit and attached with the components described herein or it can be integrally formed with any of the components. It should be understood that the coordinator component 405 is communicatively attached with the various components using any suitable mechanism or technique, a non-limiting example of which includes being wired or circuited together.

The transceiver 402 (i.e., wireless circuit) is any suitable mechanism or device that is operable for transmitting data (e.g., hemodynamic data) to an external receiver. A non-limiting example of such a device is described by “M. Ghovanloo and S. Atluri, in “A wideband power-efficient inductive wireless link for implantable microelectronic devices using multiple carriers,” IEEE Trans. on Circuits and Systems-I, vol. 54, no. 10, pp. 2211-2221, October 2007, which is incorporated by reference as though fully set forth herein.

The sensory component 404 is any suitable mechanism or device that is operable for sensing oxygen and/or other hemodynamic parameters (such as hydrostatic pressure, blood oxygen/carbon dioxide partial pressures, blood velocity (i.e., blood flow rate), blood viscosity, blood biochemistry, and cardiac chamber volume). A non-limiting example of a suitable sensory component 404 is an oxygen sensor, as described by Grist, S. M., et al., in “Oxygen Sensors for Applications in Microfluidic Cell Culture”, Sensors 2010, 10, 9286-9316, which is incorporated by reference as though fully set forth herein. Further, the sensory component 404 can be a microfluidic sensory component that detects/senses the hemodynamic parameters. Additional non-limiting examples of a suitable sensory component 404 include a magnetic probe that measure parameters with respect to blood flow, an ultrasonic probe that measure parameters with respect to blood flow, and a piezoelectric probe that measure parameters with respect to blood pressure and flow.

With respect to the cardiac chamber volume, the volume can be measured based on a flow rate. For further understanding, FIG. 4B is an illustration of a sensory component that is suitable for measuring blood flow and determining cardiac chamber volume. In this aspect, the sensory component includes a flow measurement apparatus 403 to measure the instantaneous flow based on electromagnetic properties of the blood. This flow measurement apparatus 403 includes a metering ring. A magnetic field is applied to the metering ring, which leads to a potential difference proportional to the blood flow velocity perpendicular to the flux lines. The physical principle is electromagnetic induction that works on blood as a conducting fluid.

The magnetic field penetrates the measuring ring or semi-annular ring. In line with the law of induction, a voltage V is induced in the process blood that is proportional to the flow velocity u of the blood, induction B and the internal ring diameter d, and c is a coefficient. The following expression is applicable:

V=c×B×d×u

The signal voltage “V” is detected by the sensors 407 inside the apparatus 403 that are in conductive contact with the blood. Using

q=u×π×d²/4,

the signal voltage V is converted by a signal converter into a flow parameter q, where:

q=V×π×d/(4×c×B),

and the flow parameter is then converted into standardized signals appropriate to the process.

A non-limiting example of the sensors 407 are electrodes that can be designed as capacitor plates fitted to the outside diameter or the non-conductive measuring ring-shaped apparatus. The electrodes can be formed of any suitable material that allows for good electrical contact with the passing blood, non-limiting examples of which include stainless steel, titanium, CrNi, etc, that allows good electrical contact with the blood. It should be understood that the flow measurement apparatus 403 (which is an example of a sensory component) can be incorporated any suitable cardiac device, such as a sewing ring of a heart valve, an annuluoplasty ring, an already implanted cardiovascular device (e.g., through trans-catheter procedures). Finally, as a flow measurement apparatus 403, the device can measure flow either uni-directionally and/or bi-directionally.

The flow measurement apparatus 403 can be formed in any suitable manner.

As a non-limiting example, the sensors 407 are part of a tape or other rollable item that can be rolled into a semi-circular shape and that is attachable with the cardiac device and/or other components described herein.

The energy harvesting system 406 is any suitable mechanism or device that is operable for harvesting energy and/or measuring pressure. A non-limiting example of such a device is that described by Piazza, G, et al., in “Design of a Monolithic Piezoelectrically Actuated Microelectromechanical Tunable Vertical-Cavity Surface-Emitting Laser, Optics Letters”, 2005;30:8, pp. 896-898, which is incorporated by reference as though fully set forth herein.

As another non-limiting example and as depicted in FIG. 4C, the energy harvesting system 406 is a piezoelectric sensor that generates an electrical field from blood flow pressure, harvesting approximately 5 mW. The blood flow pressure can be provided as a monitored parameter, while the power harvested can be used to operate the monitoring system. As a non-limiting example, the energy harvesting system 406 includes an energy harvesting component 408, a voltage regulator 410, and a DC-DC converter 412, which provides the electrical power to the transceiver 402. A non-limiting example of a suitable energy harvesting component 408 is piezoelectric sensor/transducer A non-limiting example of a suitable voltage regulator 410 and a non-limiting example of a suitable DC-DC converter is 412 are described by Ramadass, Y. K. and Chandrakasan, A. P, in “An Efficient Piezoelectric Energy Harvesting Interface Circuit Using a Bias-Flip Rectifier and Shared Inductor” IEEE Journal of Solid-State Circuits, January 2010 Volume: 45 Issue: 1 page189-204, which is incorporated by reference as though fully set forth herein.

In another aspect, the monitoring system includes an energy storage device that is configured to store energy, such as thermal energy from at least one of blood or body tissue and blood kinetic energy from the blood stream in the circulatory system. A battery or other energy storage component can be included to store the energy.

As described above and as illustrated in FIG. 5, the present invention allows for wireless transmission from the monitoring system 100 to a control device 500 (such as the wireless base station depicted in FIG. 3), which can simultaneously send control signals back to the monitoring system 100. In its wireless embodiment, the monitoring system 100 includes an analogue to digital converter (ADC), a digital signal processor (DSP), forward Error correction (FEC) and an RF communication transceiver (RX and TX).

Non-limiting examples of the components of the monitoring system 100 as illustrated in FIG. 5 are as follows. A suitable sensor is a 0.1 μW sensor, as described Chen, F.; Chandrakasan, A. P.; Stojanović, V.; Dept. of EECS, Massachusetts Inst. of Technol., Cambridge, Mass., USA, in “A signal-agnostic compressed sensing acquisition system for wireless and implantable sensors” 2010 IEEE Custom Integrated Circuits Conference (CICC), 19-22 September 2010 pages 1-4, which is incorporated by reference as though fully set forth herein.

An example of the analog-to digital converter (ADC) is a 5-bit, 20 KHz, 0.8 μW, 0.05 mm² @ 90 nm CMOS, 0.7 V ADC, as described by F. Chen et al, CICC 2010. An example of the RF communication transceiver is a 16 μW transceiver as described F. Chen et al, CICC 2010. Further, examples of a digital signal processor and forward error correction are those described by Arabi, K. and Sawan, M., in “A secure communication protocol for externally controlled implantable devices,” Engineering in Medicine and Biology Society, 1995., IEEE 17th Annual Conference, Issue Date: 20-23 Sep. 1995, pages 1661-1662, vol.2, on 20 Sep. 1995-23 September 1995, at Montreal, Que., Canada, which is incorporated by reference as though fully set forth herein.

As can be appreciated, transmitting signals through body tissue can have an adverse affect on signal strength and transmission. Such transmission loss can be affected by the weight of individual. In other words, the patient's body mass index (BMI) has a direct relationship with signal loss. The signal loss increases as the patient's BMI increases. For example, if BMI<18.5, a single sensor is sufficient. Alternatively, if BMI>29, multiple sensors are likely required.

The reliability of the link will be improved with the addition of error correction and with re-transmission of the data that is in error. Thus, while there has been significant research on an efficient implementation of an ADC and transceiver, the present invention improves upon the prior art by providing a forward error correction scheme and a multi-hopping algorithm which can lower transmission loss. FEC is a technique used for controlling errors in data transmission over unreliable or noisy communication channels.

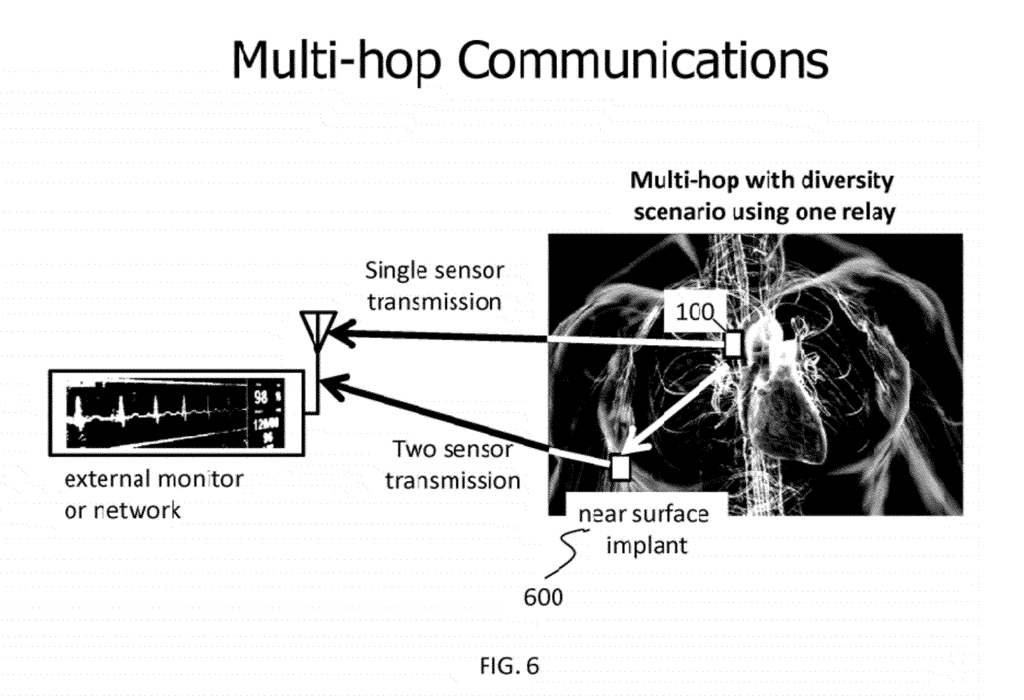

The use of multi-hop can overcome fading and reduce deep tissue implant power requirements over “single-hop” (direct transmission). For further understanding, FIG. 6 provides an illustration of a multi-hop implantation. In this example, a sensor/wireless communications device 600 (i.e., node) is implanted near the surface of the patient. The near surface implant sensor is operable for receiving a signal from the monitoring system 100 and relaying the signal to other nodes or to the external monitor or network. Although FIG. 6 illustrates a single node, it should be understood that multiple nodes can be used to relay the signal (as illustrated in FIG. 3). Thus, each intermediate node amplifies the received signal from the preceding node before retransmission. In doing so, the multi-hop implantation provides for an amplified relaying of the signal, increased diversity of the signal, and regeneration of the signal. A non-limiting example of a suitable sensor/wireless communications device that can serve as a node is that described by M. Ghovanloo and S. Atluri, in “A wideband power-efficient inductive wireless link for implantable microelectronic devices using multiple carriers,” IEEE Trans. on Circuits and Systems-I, vol. 54, no. 10, pp. 2211-2221, October 2007, which is incorporated by reference as though fully set forth herein.

Other examples of a suitable node device include those as described by the Medical Implant Communication Service (MICS), which is the name of a specification for using a frequency band between 401 and 406 MHz in communication with medical implants. It allows bi-directional radio communication with electronic implants.

In summary, the present invention is directed to an implanted wireless hemodynamic monitoring system that that can be incorporated into an existing, pre-approved heart-valve (or other cardiac device) for continuously monitoring blood flow rate, blood volume, and ventricular pressure (and other suitably monitorable parameter).

What is claimed is:

1. A wireless monitoring system comprising:
at least one sensory component adapted to measure one or more hemodynamic parameters inside a cardiac chamber of a subject; and
at least one coordinator component that receives the data from the sensory component; and
at least one transmitter communicatively attached with the coordinator component and adapted to transmit a signal containing data corresponding to the one or more hemodynamic parameters.

2. The system of claim 1, wherein the at least one sensory component is integrated within an implantable cardiac device.

3. The system of claim 1, wherein the sensory component is a microfluidic system that measures one or more hemodynamic parameter(s).

4. The system of claim 1, wherein the sensory component is a component selected from a group consisting of a magnetic probe, an ultrasonic probe, and a piezoelectric probe, and a flow measurement apparatus.

5. The system of claim 1, wherein the implantable cardiac device is a device selected from a group consisting of a heart valve, an annuloplasty ring, and a mitral valve sewing ring.

6. The system of claim 1, wherein the transmitter is a transceiver that is adapted to both transmit a signal containing data corresponding to the one or more hemodynamic parameters and receive a control signal from an external control device.

7. The system of claim 1, further comprising an energy harvesting system attached with the coordinator component.

8. The system of claim 1, further comprising an energy harvesting system attached with the coordinator component, and wherein each of the sensory component, coordinator component, transceiver, and energy harvesting system are formed as micro-fabricated ring-form components and attached with one another in a stacked configuration.

9. The system of claim 1, wherein each of the sensory component, coordinator component, transceiver, and energy harvesting system are formed as micro-fabricated components and attached with one another in a packed configuration.

10. The system of claim 1, wherein the at least one sensory component is adapted to measure a parameter selected from a group consisting of hydrostatic pressure of blood, blood gas partial pressures, blood velocity, blood viscosity, and cardiac chamber(s) volume.

11. The system of claim 1, wherein the at least one sensory component configured to measure one or more hemodynamic parameters inside a cardiac chamber of a subject measures data intermittently.

12. The system of claim 1, further comprising a plurality of sensor/wireless communications devices for implantation near the surface of the patient, the sensor/wireless communications devices being operable for receiving a signal from the monitoring system and relaying the signal to other sensor/wireless communications devices or to an external network.

13. The system of claim 1, wherein the transmitter is configured to transmit data intermittently.

14. The system of claim 1, wherein the transmitter is configured to transmit data continuously.

15. The system of claim 1, wherein the at least one sensory component is integrated within an implantable cardiac device;
wherein the sensory component is a microfluidic system that measures one or more hemodynamic parameter(s);
wherein the sensory component is a component selected from a group consisting of a magnetic probe, an ultrasonic probe, a piezoelectric probe, and a flow measurement apparatus;
wherein the implantable cardiac device is a device selected from a group consisting of a heart valve, an annuloplasty ring, and a mitral valve sewing ring;
wherein the transmitter is a transceiver that is adapted to both transmit a signal containing data corresponding to the one or more hemodynamic parameters and receive a control signal from an external control device;
further comprising an energy harvesting system attached with the coordinator component;
wherein each of the sensory component, coordinator component, transceiver, and energy harvesting system are formed in a configuration selected from a group consisting of micro-fabricated ring-form components and attached with one another in a stacked configuration and micro-fabricated components that are attached with one another in a packed configuration;
wherein the at least one sensory component is adapted to measure a parameter selected from a group consisting of hydrostatic pressure of blood, blood gas partial pressures, blood velocity, blood viscosity, and cardiac chamber(s) volume; and
further comprising a plurality of sensor/wireless communications devices for implantation near the surface of the patient, the sensor/wireless communications devices being operable for receiving a signal from the monitoring system and relaying the signal to other sensor/wireless communications devices or to an external network.
